SEQUENCE LISTING (21 sequences)

SEQ ID NO: 1 - DNA, 798 bp, Escherichia coli
atgcaggaatacacgaatcattccgataccacttttgcactgcgtaatatctcctttcgtgtgcccgggcgcacgcttttgcatccgctgtcgttaacctttcctgccgggaaagtgaccggtctgattggtcacaacggttctggtaaatccactctgctcaaaatgcttggccgtcatcagccgccgtcggaaggggagattcttcttgatgcccaaccgctggaaagctggagcagcaaagcgtttgcccgcaaagtggcttatttgccgcagcagcttcctccggcagaagggatgaccgtgcgtgaactggtggcgattggtcgttacccgtggcatggcgcgctggggcgctttggggcggcagatcgcgaaaaagtcgaggaagctatctcgctggttggcttaaaaccgctggcgcatcggctggtcgatagtctctctggcggcgaacgtcagcgggcgtggatcgccatgctggtggcgcaggatagccgttgtctgttgctcgacgaaccgacctcggcgctggatatcgcccaccaggttgatgtgctgtcgctggtgcaccgtttaagtcaggagcgtggcctgacggtcattgccgtgttgcacgatatcaatatggcggcacgctactgtgattatctggtcgccctgcgcggcggtgaaatgattgctcagggaacgcctgcggaaattatgcgcggcgaaaccctcgaaatgatttatggcatcccgatgggtattttgccgcatccggcgggtgctgcacctgtgagttttgtttattga

SEQ ID NO: 2 - DNA, 891 bp, Escherichia coli
atgagcggcttacctcttatttcgcgccgtcgactgttaacggcgatggcgctttctccgttgttatggcagatgaataccgcccacgcggcggctattgatcccaatcgtattgtggcgctggagtggttgccggtggaattactgctggcgctcggcatcgtgccttacggcgtggcggataccatcaactatcgcctgtgggtcagcgaaccaccattgccggactcagtgatcgacgtcggtttgcgcacagaacctaaccttgaactgctgaccgaaatgaaaccatcgtttatggtctggtcggcaggatatggcccttcaccagaaatgctggctcgtattgcgccgggtcgcggatttaacttcagtgacggcaaacagccgttggcgatggcgcgtaaatcgctgacggaaatggcagatttacttaacctgcaaagcgcagcggaaacgcatttagcgcaatatgaagactttatccgcagcatgaaaccccgctttgtgaagcgtggtgcgcgtccgttattgctgacgacgcttatcgatccgcgccatatgctggtcttcggtccaaacagcttgttccaggaaattcttgatgagtacggcatcccaaatgcctggcaaggggaaaccaacttctggggcagtaccgccgtcagtatcgatcgtctggcggcgtataaagacgttgatgtgctctgttttgatcacgacaacagcaaagacatggatgcgctaatggcaacgccgctgtggcaggccatgccgtttgtccgcgccggacgctttcagcgcgtacctgcagtctggttttatggtgcgacgctctcggcaatgcactttgtgcgcgttctggataacgccatcggaggtaaagcgtga

SEQ ID NO: 3 - DNA, 1983 bp, Escherichia coli
gtgagtaaacgaattgcgcttttcccggcgttattgctggcgctgttagtgattgtcgctacggcgctcacctggatgaacttctcgcaggcgctgccgcgtagccagtgggcgcaggctgcctggtcgccggatattgacgtcatcgagcagatgatttttcactacagcttgttgccgcgtctggcgatttcgctgctggtgggcgcgggtctggggctggtgggcgtgctgtttcagcaagtgctgcgtaacccgctggcggagccgacgacgcttggcgttgctacaggcgcgcaactggggattaccgtcactacgctctgggcgatccctggtgcgatggcgagccagtttgctgcgcaggcaggggcttgtgttgttggcttaattgtctttggcgtcgcgtgggggaaacggctgtcgccggtaacgctgattctcgcggggttggtagtgagcctttattgcggcgcaatcaatcagttactggttatcttccatcatgaccaactgcaaagcatgtttctgtggagcactggaacgctgacgcaaaccgactggggcggcgttgagcgtttatggccgcagctgctgggcggtgtgatgctgacgttgctgctacttcgtccgttaaccctgatggggcttgatgatggcgtggcgcgcaatctcgggctggccttgtcgcttgcgcgtctggcagcgctgtcgctggcgattgtcatcagtgcgctgctggtgaacgctgtggggattatcggctttatcgggttgttcgcgccgctgctggcaaaaatgctgggggcgcggcgtctgctgccacgactgatgctggcgtcgttgattggtgcgctgatcctctggctttccgatcaaatcatcctctggctgactcgcgtgtggatggaagtgtccaccggttcggtcactgcgttgatcggtgcgccgctgctactgtggctgttgccgcgtttacgcagcattagcgcgccggatatgaaggtcaacgatcgtgtcgcggctgaacgccaacatgtgctggcgtttgccctcgcgggcggcgtgctgctgttgatggctgtggtggtggcgctgtcgtttggtcgtgatgcgcacggctggacgtgggcgagcggggcgttgctcgaggatttaatgccctggcgctggccgcgaattatggcggcgctgtttgcgggcgtcatgctggcggtggcgggctgtattattcagcgactgaccggaaacccgatggcaagcccggaagtgctggggattagctccggcgcggcgtttggcgtggtgttgatgctgtttctggtgccgggtaatgcctttggctggctgttacctgcaggcagtctcggcgcggcggtgacgctgttgatcattatgatcgccgccggccgcggtggattttccccacaccgtatgttactggcggggatggcgttaagcaccgcgttcaccatgcttttgatgatgttgcaggcaagtggtgacccgcgaatggcgcaagtgctgacctggatttccggttcgacctacaacgcgaccgatgcgcaggtctggcgcaccggaattgtgatggtgattttgctggcgattaccccgctgtgccgccgctggctgaccattttaccgctgggtggtgataccgcccgagccgtaggaatggcgctgacgccgacgcgaattgcgctgctgctgttagcggcttgcctgacggcgaccgcgacgatgactattggaccgttgagttttgttggtttaatggcaccgcatattgcgcggatgatgggctttcgacggacgatgccacacatcgtaatttcggcgctggtgggtggtttactgctggtgttcgctgactggtgtgggcggatggtgctgtttccattccagatcccggcggggctgctgtcaacctttatcggcgcgccatattttatctatttgttgagaaagcagagccgttaa

SEQ ID NO: 4 - DNA, 3667 bp, Escherichia coli
atgcaggaatacacgaatcattccgataccacttttgcactgcgtaatatctcctttcgtgtgcccgggcgcacgcttttgcatccgctgtcgttaacctttcctgccgggaaagtgaccggtctgattggtcacaacggttctggtaaatccactctgctcaaaatgcttggccgtcatcagccgccgtcggaaggggagattcttcttgatgcccaaccgctggaaagctggagcagcaaagcgtttgcccgcaaagtggcttatttgccgcagcagcttcctccggcagaagggatgaccgtgcgtgaactggtggcgattggtcgttacccgtggcatggcgcgctggggcgctttggggcggcagatcgcgaaaaagtcgaggaagctatctcgctggttggcttaaaaccgctggcgcatcggctggtcgatagtctctctggcggcgaacgtcagcgggcgtggatcgccatgctggtggcgcaggatagccgttgtctgttgctcgacgaaccgacctcggcgctggatatcgcccaccaggttgatgtgctgtcgctggtgcaccgtttaagtcaggagcgtggcctgacggtcattgccgtgttgcacgatatcaatatggcggcacgctactgtgattatctggtcgccctgcgcggcggtgaaatgattgctcagggaacgcctgcggaaattatgcgcggcgaaaccctcgaaatgatttatggcatcccgatgggtattttgccgcatccggcgggtgctgcacctgtgagttttgtttattgatgagcggcttacctcttatttcgcgccgtcgactgttaacggcgatggcgctttctccgttgttatggcagatgaataccgcccacgcggcggctattgatcccaatcgtattgtggcgctggagtggttgccggtggaattactgctggcgctcggcatcgtgccttacggcgtggcggataccatcaactatcgcctgtgggtcagcgaaccaccattgccggactcagtgatcgacgtcggtttgcgcacagaacctaaccttgaactgctgaccgaaatgaaaccatcgtttatggtctggtcggcaggatatggcccttcaccagaaatgctggctcgtattgcgccgggtcgcggatttaacttcagtgacggcaaacagccgttggcgatggcgcgtaaatcgctgacggaaatggcagatttacttaacctgcaaagcgcagcggaaacgcatttagcgcaatatgaagactttatccgcagcatgaaaccccgctttgtgaagcgtggtgcgcgtccgttattgctgacgacgcttatcgatccgcgccatatgctggtcttcggtccaaacagcttgttccaggaaattcttgatgagtacggcatcccaaatgcctggcaaggggaaaccaacttctggggcagtaccgccgtcagtatcgatcgtctggcggcgtataaagacgttgatgtgctctgttttgatcacgacaacagcaaagacatggatgcgctaatggcaacgccgctgtggcaggccatgccgtttgtccgcgccggacgctttcagcgcgtacctgcagtctggttttatggtgcgacgctctcggcaatgcactttgtgcgcgttctggataacgccatcggaggtaaagcgtgagtaaacgaattgcgcttttcccggcgttattgctggcgctgttagtgattgtcgctacggcgctcacctggatgaacttctcgcaggcgctgccgcgtagccagtgggcgcaggctgcctggtcgccggatattgacgtcatcgagcagatgatttttcactacagcttgttgccgcgtctggcgatttcgctgctggtgggcgcgggtctggggctggtgggcgtgctgtttcagcaagtgctgcgtaacccgctggcggagccgacgacgcttggcgttgctacaggcgcgcaactggggattaccgtcactacgctctgggcgatccctggtgcgatggcgagccagtttgctgcgcaggcaggggcttgtgttgttggcttaattgtctttggcgtcgcgtgggggaaacggctgtcgccggtaacgctgattctcgcggggttggtagtgagcctttattgcggcgcaatcaatcagttactggttatcttccatcatgaccaactgcaaagcatgtttctgtggagcactggaacgctgacgcaaaccgactggggcggcgttgagcgtttatggccgcagctgctgggcggtgtgatgctgacgttgctgctacttcgtccgttaaccctgatggggcttgatgatggcgtggcgcgcaatctcgggctggccttgtcgcttgcgcgtctggcagcgctgtcgctggcgattgtcatcagtgcgctgctggtgaacgctgtggggattatcggctttatcgggttgttcgcgccgctgctggcaaaaatgctgggggcgcggcgtctgctgccacgactgatgctggcgtcgttgattggtgcgctgatcctctggctttccgatcaaatcatcctctggctgactcgcgtgtggatggaagtgtccaccggttcggtcactgcgttgatcggtgcgccgctgctactgtggctgttgccgcgtttacgcagcattagcgcgccggatatgaaggtcaacgatcgtgtcgcggctgaacgccaacatgtgctggcgtttgccctcgcgggcggcgtgctgctgttgatggctgtggtggtggcgctgtcgtttggtcgtgatgcgcacggctggacgtgggcgagcggggcgttgctcgaggatttaatgccctggcgctggccgcgaattatggcggcgctgtttgcgggcgtcatgctggcggtggcgggctgtattattcagcgactgaccggaaacccgatggcaagcccggaagtgctggggattagctccggcgcggcgtttggcgtggtgttgatgctgtttctggtgccgggtaatgcctttggctggctgttacctgcaggcagtctcggcgcggcggtgacgctgttgatcattatgatcgccgccggccgcggtggattttccccacaccgtatgttactggcggggatggcgttaagcaccgcgttcaccatgcttttgatgatgttgcaggcaagtggtgacccgcgaatggcgcaagtgctgacctggatttccggttcgacctacaacgcgaccgatgcgcaggtctggcgcaccggaattgtgatggtgattttgctggcgattaccccgctgtgccgccgctggctgaccattttaccgctgggtggtgataccgcccgagccgtaggaatggcgctgacgccgacgcgaattgcgctgctgctgttagcggcttgcctgacggcgaccgcgacgatgactattggaccgttgagttttgttggtttaatggcaccgcatattgcgcggatgatgggctttcgacggacgatgccacacatcgtaatttcggcgctggtgggtggtttactgctggtgttcgctgactggtgtgggcggatggtgctgtttccattccagatcccggcggggctgctgtcaacctttatcggcgcgccatattttatctatttgttgagaaagcagagccgttaa

SEQ ID NO: 5 - protein, 265 aa, Escherichia coli
MQEYTNHSDTTFALRNISFRVPGRTLLHPLSLTFPAGKVTGLIGHNGSGKSTLLKMLGRHQPPSEGEILLDAQPLESWSSKAFARKVAYLPQQLPPAEGMTVRELVAIGRYPWHGALGRFGAADREKVEEAISLVGLKPLAHRLVDSLSGGERQRAWIAMLVAQDSRCLLLDEPTSALDIAHQVDVLSLVHRLSQERGLTVIAVLHDINMAARYCDYLVALRGGEMIAQGTPAEIMRGETLEMIYGIPMGILPHPAGAAPVSFVY

SEQ ID NO: 6 - protein, 296 aa, Escherichia coli
MSGLPLISRRRLLTAMALSPLLWQMNTAHAAAIDPNRIVALEWLPVELLLALGIVPYGVADTINYRLWVSEPPLPDSVIDVGLRTEPNLELLTEMKPSFMVWSAGYGPSPEMLARIAPGRGFNFSDGKQPLAMARKSLTEMADLLNLQSAAETHLAQYEDFIRSMKPRFVKRGARPLLLTTLIDPRHMLVFGPNSLFQEILDEYGIPNAWQGETNFWGSTAVSIDRLAAYKDVDVLCFDHDNSKDMDALMATPLWQAMPFVRAGRFQRVPAVWFYGATLSAMHFVRVLDNAIGGKA

SEQ ID NO: 7 - protein, 660 aa, Escherichia coli
MSKRIALFPALLLALLVIVATALTWMNFSQALPRSQWAQAAWSPDIDVIEQMIFHYSLLPRLAISLLVGAGLGLVGVLFQQVLRNPLAEPTTLGVATGAQLGITVTTLWAIPGAMASQFAAQAGACVVGLIVFGVAWGKRLSPVTLILAGLVVSLYCGAINQLLVIFHHDQLQSMFLWSTGTLTQTDWGGVERLWPQLLGGVMLTLLLLRPLTLMGLDDGVARNLGLALSLARLAALSLAIVISALLVNAVGIIGFIGLFAPLLAKMLGARRLLPRLMLASLIGALILWLSDQIILWLTRVWMEVSTGSVTALIGAPLLLWLLPRLRSISAPDMKVNDRVAAERQHVLAFALAGGVLLLMAVVVALSFGRDAHGWTWASGALLEDLMPWRWPRIMAALFAGVMLAVAGCIIQRLTGNPMASPEVLGISSGAAFGVVLMLFLVPGNAFGWLLPAGSLGAAVTLLIIMIAAGRGGFSPHRMLLAGMALSTAFTMLLMMLQASGDPRMAQVLTWISGSTYNATDAQVWRTGIVMVILLAITPLCRRWLTILPLGGDTARAVGMALTPTRIALLLLAACLTATATMTIGPLSFVGLMAPHIARMMGFRRTMPHIVISALVGGLLLVFADWCGRMVLFPFQIPAGLLSTFIGAPYFIYLLRKQSR

SEQ ID NO: 8 - DNA, 70 bp, Artificial Sequence
  Primer 1
ctcctttcgtgtgcccgggcgcacgcttttgcatccgctgtcgttaacctaggtgacactatagaacgcg

SEQ ID NO: 9 - DNA, 70 bp, Artificial Sequence
  Primer 2
caataaacaaaactcacaggtgcagcacccgccggatgcggcaaaatacctagtggatctgatgggtacc

SEQ ID NO: 10 - DNA, 70 bp, Artificial Sequence
  Primer 3
cgactgttaacggcgatggcgctttctccgttgttatggcagatgaatacaggtgacactatagaacgcg

SEQ ID NO: 11 - DNA, 70 bp, Artificial Sequence
  Primer 4
tcacgctttacctccgatggcgttatccagaacgcgcacaaagtgcattgtagtggatctgatgggtacc

SEQ ID NO: 12 - DNA, 70 bp, Artificial Sequence
  Primer 5
gtgagtaaacgaattgcgcttttcccggcgttattgctggcgctgttagtaggtgacactatagaacgcg

SEQ ID NO: 13 - DNA, 70 bp, Artificial Sequence
  Primer 6
aggttgacagcagccccgccgggatctggaatggaaacagcaccatccgctagtggatctgatgggtacc

SEQ ID NO: 14 - DNA, 70 bp, Artificial Sequence
  Primer 7
atgcaggaatacacgaatcattccgataccacttttgcactgcgtaatatctcctttcgtgtgcccgggc

SEQ ID NO: 15 - DNA, 70 bp, Artificial Sequence
  Primer 8
gccatcgccgttaacagtcgacggcgcgaaataagaggtaagccgctcatcaataaacaaaactcacagg

SEQ ID NO: 16 - DNA, 70 bp, Artificial Sequence
  Primer 9
cctgtgagttttgtttattgatgagcggcttacctcttatttcgcgccgtcgactgttaacggcgatggc

SEQ ID NO: 17 - DNA, 70 bp, Artificial Sequence
  Primer 10
aatcactaacagcgccagcaataacgccgggaaaagcgcaattcgtttactcacgctttacctccgatgg

SEQ ID NO: 18 - DNA, 70 bp, Artificial Sequence
  Primer 11
tcggcaatgcactttgtgcgcgttctggataacgccatcggaggtaaagcgtgagtaaacgaattgcgct

SEQ ID NO: 19 - DNA, 70 bp, Artificial Sequence
  Primer 12
ttaacggctctgctttctcaacaaatagataaaatatggcgcgccgataaaggttgacagcagccccgcc

SEQ ID NO: 20 - DNA, 26 bp, Artificial Sequence
  Primer 13
gaatacgtcgccagctgctttaacac

SEQ ID NO: 21 - DNA, 24 bp, Artificial Sequence
  Primer 14
tggtttgtcggatgcggcgtgaac

(19) United States
(12) Patent Application Publication
(10) Pub. No.: US 2017/0081633 A1
(43) Pub. Date: Mar. 23, 2017

(54) MICROORGANISM HAVING IMPROVED INTRACELLULAR ENERGY LEVEL AND METHOD FOR PRODUCING L-AMINO ACID USING SAME

(71) Applicant: CJ CHEILJEDANG CORPORATION, Seoul (KR)

(72) Inventors: Juno JANG, Seoul (KR); Hye Min PARK, Seoul (KR); Kwang Ho LEE, Seoul (KR); Keun Cheol LEE, Hwaseong-si, Gyeonggi-do (KR); Hyeong Pyo HONG, Gangneung-si, Gangwon-do (KR)

(21) Appl. No.: 15/312,497

(22) PCT Filed: Apr. 14, 2015
(86) PCT No.: PCT/KR2015/003625

§ 371 Date: Nov. 18, 2016

(30) Foreign Application Priority Data
May 23, 2014 (KR) 10-2014-0062593

(51) Int. Cl. C12N 1/20; C12P 13/22

(52) U.S. Cl. CPC C12N 1/20; C12P 13/227

(57) ABSTRACT

The present application relates to a recombinant microorganism having an improved intracellular energy level and a method for producing L-amino acid using the microorganism.

### TECHNICAL FIELD

The present application relates to a recombinant microorganism having an improved intracellular energy level and a method for producing L-amino acids using the microorganism.

### BACKGROUND ART

For production of a desired material using a microorganism, desired material-specific approaches have been mainly used, such as enhancement of expression of genes encoding enzymes involved in the production of the desired material or removal of unnecessary genes. For example, a number of useful strains including E. coli capable of producing a desired L-amino acid with a high yield have been developed by enhancement of a biosynthetic pathway of the L-amino acid. High-yield production of useful desired materials using microorganisms requires production and maintenance of sufficient energy.

For In vivo biosynthesis of materials such as proteins, nucleic acids, etc., energy conserved in the form of NADH, NADPH and ATP (Adenosine-5′-triphosphate) is used. Particularly, ATP is an energy carrier that transports chemical energy produced in metabolic reactions to various activities of organisms.

ATP is mainly produced in metabolic processes of microorganisms. Major intracellular ATP production pathways are substrate level phosphorylation that takes place via glycolysis or oxidative phosphorylation that produces ATP through the electron transport system using a reducing power accumulated in NADH, etc. via glycolysis. The generated ATP is consumed in vivo activities such as biosynthesis, motion, signal transduction, and cell division. Therefore, industrial microorganisms used for the production of useful desired materials generally exhibit high ATP demand. Accordingly, studies have been conducted to improve productivity by increasing intracellular energy levels upon mass-production of useful desired materials (Biotechnol Adv (2009) 27:94-101).

Iron is one of elements essential for maintenance of homeostasis of microorganisms, and E. coli utilizes various routes for uptake of iron (Mol Microbiol (2006) 62:120-131). One of the iron uptake routes is to uptake iron via FhuCDB complex channels formed by FhuC, FhuD, and FhuB proteins. Recently, it was revealed that in the presence of excess L-tryptophan in cells, TrpR protein regulating expression of genes involved in L-tryptophan biosynthesis forms a complex with L-tryptophan, and in turn, this complex binds to a regulatory region of fhuCDB operon, suggesting the possibility of a correlation between iron uptake via the FhuCDB protein complex and L-tryptophan biosynthesis. However, function of the FhuCDB protein complex in L-tryptophan biosynthesis, and its effect on iron uptake have not been clarified yet (Nat Chem Biol (2012) 8:65-71).

The present inventors have studied methods of improving ATP levels and increasing producibility of useful desired materials such as L-amino acids, and they found that intracellular ATP levels may be improved by inactivation of the function of the FhuCDB protein complex by deletion of fhuCDB gene, and as a result, producibility of desired materials may be increased, thereby completing the present application.

### DETAILED DESCRIPTION OF THE INVENTION

### Technical Problem

An object of the present application is to provide a microorganism having an improved intracellular ATP level.

Another object of the present application is to provide a method for producing a desired material using the microorganism having the improved intracellular ATP level.

### Technical Solution

In an aspect, the present application provides a microorganism having an improved intracellular ATP level.

In a specific embodiment of the present application, the microorganism may be a microorganism in which activities of one or more of iron uptake system-constituting protein FhuC, protein FhuD, and protein FhuB were inactivated, and therefore, has an increased intracellular ATP level, compared to an unmodified strain.

The term “FhuCDB”, as used herein, is a component of an iron uptake system (fhu system) which includes expression products of fhuA, fhuC, fhuD and fhuB arranged in one operon. The fhuA encodes multi-functional OMP FhuA (79 kDa) which acts as a receptor for ferrichrome-iron, phages, bacterial toxins, and antibiotics. FhuA is specific to Fe³⁺-ferrichrome, and acts as a ligand-specific gated channel (Protein Sci 7, 1636-1638). The other proteins of the fhu system, namely, FhuD, FhuC and FhuB are also essential for the functions of the iron uptake system. A periplasmic protein, FhuD and cytoplasmic membrane-associated proteins, FhuC and FhuB form a FhuCDB complex, which functions to transport ferrichrome and other Fe³⁺-hydroxamate compounds (Fe³⁺-aerobactin, Fe³⁺-coprogen) across the cytoplasmic membrane from the periplasm into the cytoplasm (J Bacteriol 169, 3844-3849). Uptake of iron via the FhuCDB complex consumes one molecule of ATP, and for this iron uptake process, a protein complex, TonB-ExbB-ExbD provides energy (FEBS Lett 274, 85-88).

FhuC encodes a cytoplasmic membrane-associated protein of 29 kDa, and forms a channel for iron uptake, together with FhuD and FhuB. FhuC may have an amino acid sequence of SEQ ID NO: 5, and specifically, FhuC may be encoded by a nucleotide sequence of SEQ ID NO: 1.

FhuD encodes a cytoplasmic membrane-associated protein of 31 kDa, and forms a channel for iron uptake, together with FhuC and FhuB. FhuD may have an amino acid sequence of SEQ ID NO: 6, and specifically, FhuD may be encoded by a nucleotide sequence of SEQ ID NO: 2.

FhuB encodes a cytoplasmic membrane-associated protein of 41 kDa, and forms a channel for iron uptake, together with FhuC and FhuB. FhuB may have an amino acid sequence of SEQ ID NO: 7, and specifically, FhuB may be encoded by a nucleotide sequence of SEQ ID NO: 3.

Specifically, although proteins have identical activities, there are small differences in the amino acid sequences between subjects. Therefore, FuhC, FhuD, and FhuB may have SEQ ID NOs: 5, 6, and 7, respectively, but are not limited thereto. That is, FuhC, FhuD, and FhuB in the present application may be variants having amino acid sequences having substitution, deletion, insertion, addition or inversion of one or more amino acids at one or more positions of the amino acid sequences, and they may have sequences having 70% or higher, 80% or higher, 90% or higher, or 95% or higher homology with the amino acid sequences of SEQ ID NOs: 5, 6, and 7, respectively. Further, in the nucleotide sequences, various modifications may be made in the coding region provided that they do not change the amino acid sequences of the proteins expressed from the coding region, due to codon degeneracy or in consideration of the codons preferred by the organism in which they are to be expressed. The above-described nucleotide sequence is provided only as an example of various nucleotide sequences made by a method well known to those skilled in the art, but is not limited thereto.

The term “homology”, as used herein, refers to a degree of identity between bases or amino acid residues after both sequences are aligned so as to best match in certain comparable regions in an amino acid or nucleotide sequence of a gene encoding a protein. If the homology is sufficiently high, expression products of the corresponding genes may have identical or similar activity. The percentage of the sequence identity may be determined using a known sequence comparison program, for example, BLASTN (NCBI), CLC Main Workbench (CLC bio), MegAlign™ (DNASTAR Inc), etc.

The term “microorganism”, as used herein, refers to a prokaryotic microorganism or a eukaryotic microorganism having an ability to produce a useful desired material such as L-amino acids. For example, the microorganism having the improved intracellular ATP level may be the genus Escherichia, the genus Erwinia, the genus Serratia, the genus Providencia, the genus Corynebacteria, the genus Pseudomonas, the genus Leptospira, the genus Salmonellar, the genus Brevibacteria, the genus Hypomononas, the genus Chromobacterium, or the genus Norcardia microorganisms or fungi or yeasts. Specifically, the microorganism may be the genus Escherichia microorganism, and more specifically, the microorganism may be E. coli.

The “unmodified strain”, as used herein, refers to a microorganism which is not modified by a molecular biological technique such as mutation or recombination. Specifically, the unmodified strain refers to a microorganism before increasing the intracellular ATP level, in which the intracellular ATP level is increased by inactivating one or more of FhuC, FhuD, and FhuB constituting the iron uptake system, FhuCDB complex, thereby having a reduction of intracellular ATP consumption. That is, the unmodified strain refers to an original microorganism from which the recombinant microorganism is derived.

In a specific embodiment of the present application, the microorganism may include inactivation of one or more of FhuC, FhuD, and FhuB, and inactivation of a combination of FhuC, FhuD, and FhuB, and specifically, inactivation of all of FhuC, FhuD, and FhuB.

The term “inactivation”, as used herein, means that the activity of the corresponding protein is eliminated or weakened by mutation due to deletion, substitution, or insertion of part or all of the gene encoding the corresponding protein, by modification of an expression regulatory sequence to reduce the expression of the gene, by modification of the chromosomal gene sequence to weaken or eliminate the activity of the protein, or by combinations thereof.

Specifically, deletion of part or all of the gene encoding the protein may be performed by replacing a polynucleotide which encodes an endogenous target protein in the chromosome, with either a polynucleotide of which a partial sequence is deleted or a marker gene through a bacterial chromosome insertion vector. Further, a mutation may be induced using a mutagen such as chemicals or UV light, thereby obtaining a mutant having deletion of the corresponding gene, but is not limited thereto.

The term “expression regulatory sequence”, as used herein, a nucleotide sequence regulating a gene expression, refers to a segment capable of increasing or decreasing expression of a particular gene in a subject, and may include a promoter, a transcription factor binding site, a ribosome-binding site, a sequence regulating the termination of transcription and translation, but is not limited thereto.

Specifically, modification of the expression regulatory sequence for causing a decrease in gene expression may be performed by inducing mutations in the expression regulatory sequence through deletion, insertion, conservative or non-conservative substitution of nucleotide sequence or a combination thereof to further weaken the activity of the expression regulatory sequence, or by replacing the expression regulatory sequence with of the sequence having weaker activity, but is not limited thereto.

In a specific embodiment of the present application, the microorganism may be a microorganism of the genus Escherichia having an improved producibility of a desired material, compared to an unmodified strain. In the microorganism of the genus Escherichia of the present application, one or more of proteins constituting FhuCDB complex are inactivated to inactivate the iron uptake pathway, and therefore, iron uptake via this pathway reduces ATP consumption. As a result, the microorganism of the genus Escherichia has an improved intracellular ATP level, compared to the unmodified strain, and consequently, the microorganism has the improved producibility of the desired material.

The term “microorganism having the improved producibility” refers to a microorganism having an improved producibility of a desired material, compared to an unmodified strain or a parent cell before modification.

The term “desired material”, as used herein, includes a material of which production amount is increased by increasing intracellular ATP level of the microorganism, without limitation. The desired material may be specifically L-amino acid, and more specifically, L-threonine or L-tryptophan.

In a specific embodiment, the microorganism may be E. coli having an improved producibility of L-tryptophan, wherein one or more of FhuC, FhuD, and FhuB from E. coli having a producibility of L-tryptophan were inactivated, and having improved intracellular ATP level, compared to an unmodified strain. The E. coli having the producibility of L-tryptophan may be obtained by increasing expression of an L-tryptophan operon gene, removing feedback inhibition by a final product L-tryptophan, or removing inhibition and attenuation of the L-tryptophan operon gene at a transcriptional level, but is not limited thereto.

In a specific embodiment of the present application, the microorganism may be E. coli having an improved producibility of L-threonine, wherein one or more of FhuC, FhuD, and FhuB from E. coli having a producibility of L-threonine were inactivated, and having improved intracellular ATP level, compared to an unmodified strain. The E. coli having the producibility of L-threonine may be obtained by increasing expression of an L-threonine operon gene, removing feedback inhibition by a final product L-threonine, or removing inhibition and attenuation of the L-threonine operon gene at a transcriptional level, but is not limited thereto.

In another aspect, the present application provides a method for producing L-amino acids, the method including culturing the microorganism of the genus Escherichia having the improved intracellular ATP level in a media, and recovering L-amino acids from the culture media or the microorganism.

The term “microorganism of the genus Escherichia having the improved intracellular ATP level”, as used herein, is the same as described above.

In the method for producing L-amino acids according to a specific embodiment of the present application, the culturing of the microorganism having the producibility of L-amino acids may be performed according to an appropriate medium and culture conditions known in the art. The culturing procedures may be readily adjusted by those skilled in the art according to the selected microorganism. Examples of the culturing procedures include batch type, continuous type and fed-batch type, but are not limited thereto.

A medium used for the culturing must meet the requirements for the culturing of a specific microorganism. The culture media for various microorganisms are described in a literature (“Manual of Methods for General Bacteriology” by the American Society for Bacteriology, Washington D.C., USA, 1981.). These media include a variety of carbon sources, nitrogen sources, and trace elements. The carbon source includes carbohydrates such as glucose, lactose, sucrose, fructose, maltose, starch and cellulose; lipids such as soybean oil, sunflower oil, castor oil and coconut oil; fatty acids such as palmitic acid, stearic acid, and linoleic acid; alcohols such as glycerol and ethanol; and organic acids such as acetic acid. These carbon sources may be used alone or in combination, but are not limited thereto. The nitrogen source includes organic nitrogen sources, such as peptone, yeast extract, gravy, malt extract, corn steep liquor (CSL) and bean flour, and inorganic nitrogen sources such as urea, ammonium sulfate, ammonium chloride, ammonium phosphate, ammonium carbonate and ammonium nitrate. These nitrogen sources may be used alone or in combination, but are not limited thereto. Additionally, the medium may include potassium dihydrogen phosphate, dipotassium hydrogen phosphate, and corresponding sodium-containing salts thereof as a phosphorus source, but is not limited thereto. Also, the medium may include a metal such as magnesium sulfate or iron sulfate. In addition, amino acids, vitamins and proper precursors may be added as well.

Further, to maintain the culture under aerobic conditions, oxygen or oxygen-containing gas (e.g., air) may be injected into the culture. A temperature of the culture may be generally 20° C.-45° C., and specifically 25° C.-40° C. The culturing may be continued until production of L-amino acids such as L-threonine or L-tryptophan reaches a desired level, and specifically, a culturing time may be 10 hrs-100 hrs.

The method for producing L-amino acids according to a specific embodiment of the present application may further include recovering L-amino acids from the culture media or the microorganism thus obtained. Recovering of L-amino acids may be performed by a proper method known in the art, depending on the method of culturing the microorganism of the present application, for example, batch type, continuous type or fed-batch type, so as to purify or recover the desired L-amino acids from the culture of the microorganism, but is not limited thereto.

### Advantageous Effects of the Invention

According to the present application, when a microorganism of the genus Escherichia having an improved intracellular ATP level, compared to an unmodified strain, and a method for producing a desired material using the same are used, the high intracellular ATP level enhances gene expression, biosynthesis, transport of materials, etc., thereby efficiently producing the useful desired material including proteins, L-amino acids, etc.

### DESCRIPTION OF THE DRAWINGS

FIG. 1 shows intracellular ATP levels of E. coli according to a specific embodiment of the present application, compared to an unmodified strain;

FIG. 2 shows intracellular ATP levels of wild-type-derived E. coli having a producibility of L-tryptophan according to a specific embodiment of the present application, compared to an unmodified strain;

FIG. 3 shows intracellular ATP levels of E. coli having a producibility of L-threonine according to a specific embodiment of the present application, compared to an unmodified strain;

FIG. 4 shows intracellular ATP levels of E. coli having a producibility of L-tryptophan according to a specific embodiment of the present application, compared to an unmodified strain;

FIG. 5 shows L-threonine producibility of E. coli having a producibility of L-threonine according to a specific embodiment of the present application, compared to an unmodified strain; and

FIG. 6 shows L-tryptophan producibility of E. coli having a producibility of L-tryptophan according to a specific embodiment of the present application, compared to an unmodified strain.

### MODE OF THE INVENTION

Hereinafter, the present application will be described in more detail with reference to Examples. However, these Examples are for illustrative purposes only, and the scope of the present application is not intended to be limited by these Examples.

### Example 1

### Preparation of Wild-Type E. coli W3110 Having Inactivation of Proteins Encoded by fhuC, fhuD, and fhuB Genes

In this Example, fhuC, fhuD, and fhuB genes of wild-type E. coli W3110 (ATCC® 39936™) were deleted by homologous recombination, respectively.

The fhuC, fhuD, and fhuB genes to be deleted have nucleotide sequences of SEQ ID NOs: 1, 2, and 3, respectively and these genes exist in the form of operon of SEQ ID NO: 4.

To delete fhuC, fhuD, and fhuB, one-step inactivation using lambda Red recombinase developed by Datsenko K A, et al. was performed (Proc Natl Acad Sci USA., (2000) 97:6640-6645). As a marker to confirm the insertion into the gene, a chloramphenicol gene of pUCprmfmloxC, which was prepared by ligating an rmf promoter to pUC19 (New England Biolabs (USA)) and ligating a mutated loxP-CmR-loxP cassette obtained from pACYC184 (New England Biolab) thereto, was used (Korean Patent Application NO. 2009-0075549).

First, primary polymerase chain reaction (hereinbelow, referred to as ‘PCR’) was performed using pUCprmfmloxC as a template and primer combinations of SEQ ID NOs: 8 and 9, 10 and 11, 12 and 13, and 8 and 13 having a part of the fhuC and fhuB genes and a partial sequence of the chloramphenicol resistant gene of the pUCprmfmloxC gene under the conditions of 30 cycles of denaturation at 94° C. for 30 seconds, annealing at 55° C. for 30 seconds and elongation at 72° C. for 1 minute, thereby obtaining PCR products of about 1.2 kb, ΔfhuC1st, ΔfhuD1st, ΔfhuB1st, and ΔfhuCDB1st.

Thereafter, the PCR products of 1.2 kb, ΔfhuC1st, ΔfhuD1st, ΔfhuB1st, and ΔfhuCDB1st obtained by PCR were electrophoresed on a 0.8% agarose gel, and then eluted and used as a template for secondary PCR. The secondary PCR was performed using the eluted primary PCR products as templates and the primer combinations of SEQ ID NOs: 14 and 15, 16 and 17, 18 and 19, 14 and 19 containing nucleotide sequences of 20 bp of the 5′ and 3′ regions of the PCR products obtained in the primary PCR under the conditions of 30 cycles of denaturation at 94° C. for 30 seconds, annealing at 55° C. for 30 seconds and elongation at 72° C. for 1 minute, thereby obtaining PCR products of about 1.3 kb, ΔfhuC, ΔfhuD, ΔfhuB, and ΔfhuCDB. The PCR products thus obtained was electrophoresed on a 0.8% agarose gel, and then eluted, and used in recombination.

E. coli W3110, which was transformed with a pKD46 vector according to the one-step inactivation method developed by Datsenko K A et al. (Proc Natl Acad Sci USA., (2000) 97:6640-6645), was prepared as a competent strain, and transformation was performed by introducing the gene fragment of 1.3 kb obtained by primary and secondary PCR. The strains were cultured on the LB medium supplemented with chloramphenicol and transformants having chloramphenicol resistance were selected. Deletion of any or all of fhuC, fhuD, and fhuB was confirmed by PCR products of about 4.4 kb, about 4.3 kb, about 3.3 kb, and about 1.6 kb which were amplified by PCR using genomes obtained from the selected strains as templates and primers of SEQ ID NOs: 20 and 21.

After removal of pKD46 from the primary recombinant strains having chloramphenicol resistance thus obtained, a pJW168 vector (Gene, (2000) 247, 255-264) was introduced into the primary recombinant strains having chloramphenicol resistance so as to remove the chloramphenicol marker gene from the strains (Gene, (2000) 247, 255-264). PCR was performed using primers of SEQ ID NOs: 20 and 21 to obtain PCR products of about 3.4 kb, about 3.3 kb, about 2.2 kb, and about 0.6 kb, indicating that the strains finally obtained had deletion of any or all of fhuC, fhuD, and fhuB genes. The strains were designated as E. coli W3110_ΔfhuC, W3110_ΔfhuD, W3110_ΔfhuB and W3110_ΔfhuCDB, respectively.

### Example 2

### Measurement of Intracellular ATP Levels in Wild-Type E. coli-Derived fhuC, fhuD, and fhuB Gene-Deleted E. coli

In this Example, the intracellular ATP levels in the strains prepared in Example 1 were practically measured.

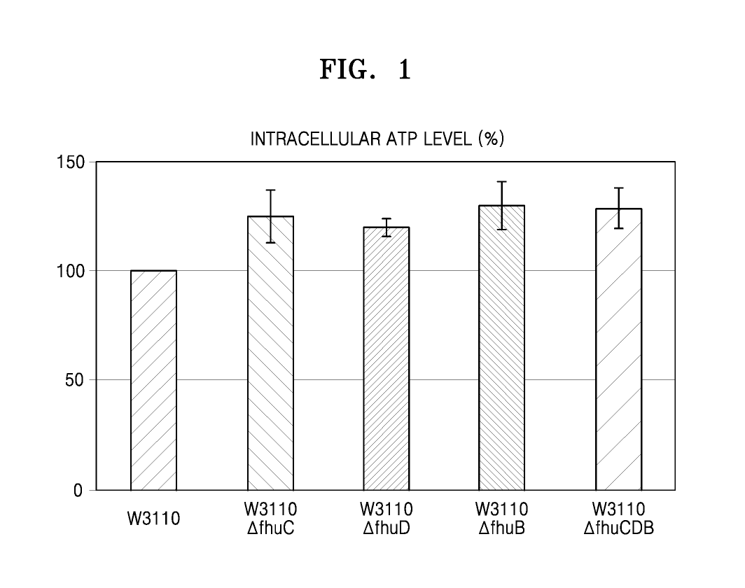

For this purpose, “An Efficient Method for Quantitative determination of Cellular ATP Synthetic Activity” developed by Kiyotaka Y. Hara et al., in which luciferase is used, was employed (J Biom Scre, (2006) Vol. 11, No. 3, pp 310-17). Briefly, E. coli W3110 which is an unmodified strain used in Example 1 and E. coli W3110_ΔfhuCDB obtained by gene deletion were cultured overnight in LB liquid medium containing glucose, respectively. After culturing, supernatants were removed by centrifugation, the cells thus obtained were washed with 100 mM Tris-Cl (pH 7.5), and then treated with PB buffer (permeable buffer: 40% [v/v] glucose, 0.8% [v/v] Triton X-100) for 30 minutes to release intracellular ATP from the cells. Next, supernatants were removed by centrifugation, and luciferin as a substrate for luciferase was added to the cells. The cells were allowed to react for 10 minutes. Color development by luciferase was measured using a luminometer to quantitatively determine ATP levels. The results are given in FIG. 1. The results of FIG. 1 were recorded as the average of three repeated experiments.

As shown in FIG. 1, the intracellular ATP levels in E. coli W3110_ΔfhuC, W3110_ΔfhuD, W3110_ΔfhuB and W3110_ΔfhuCDB prepared in Example 1, in which any or all of wild-type E. coli-derived fhuC, fhuD, and fhuB was/were deleted, were increased, compared to the unmodified strain, E. coli W3110.

### Example 3

### Preparation of Wild-Type-Derived L-Tryptophan-Producing Strain Having Inactivation of Proteins Encoded by fhuC, fhuD, and fhuB Genes and Measurement of Intracellular ATP Levels

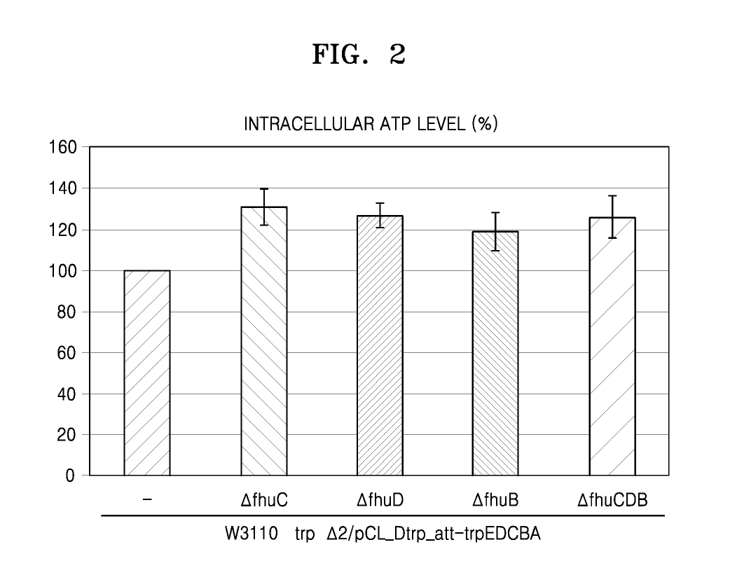

In this Example, any or all of fhuC, fhuD, and fhuB genes of a wild-type-derived L-tryptophan-producing strain, E. coli W3110 trpΔ2/pCL-Dtrp_att-trpEDCBA (Korean Patent Publication No. 10-2013-0082121) was/were deleted by homologous recombination as in Example 1 to prepare W3110 trpΔ2_ΔfhuC/pCL-Dtrp_att-trpEDCBA, W3110 trpΔ2_ΔfhuD/pCL-Dtrp_att-trpEDCBA, W3110 trpΔ2_ΔfhuB/pCL-Dtrp_att-trpEDCBA, and W3110 trpΔ2_ΔfhuCDB/pCL-Dtrp_att-trpEDCBA strains. In these strains thus prepared, intracellular ATP levels were measured in the same manner as in Example 2 and the results are given in FIG. 2.

As shown in FIG. 2, the intracellular ATP levels in the strains, which were prepared by deletion of any or all of fhuC, fhuD, and fhuB genes in the wild-type L-tryptophan-producing strain, were increased, compared to an unmodified strain and a control strain.

### Example 4

### Examination of Titer of Wild-Type-Derived L-Tryptophan-Producing Strain Having Inactivation of Proteins Encoded by fhuC, fhuD, and fhuB Genes

As described in Example 3, the wild-type-derived L-tryptophan-producing strain, W3110 trpΔ2/pCL-Dtrp_att-trpEDCBA and the strains with improved intracellular ATP levels prepared by deletion of any or all of fhuC, fhuD, and fhuB genes were subjected to titration using glucose as a carbon source.

Each of the strains was inoculated by a platinum loop on an LB solid medium, and cultured in an incubator at 37° C. overnight, and then inoculated by a platinum loop into 25 mL of a glucose-containing titration medium containing a composition of Table 1. Then, the strains were cultured in an incubator at 37° C. and at 200 rpm for 48 hours. The results are given in Table 2. All the results were recorded as the average of three repeated experiments.

TABLE 1

| | Concentration | |
|---|---|---|
| Composition | (per liter) | |
| Glucose | 60 | g |
| K₂HPO₄ | 1 | g |
| (NH₄)₂SO₄ | 15 | g |
| NaCl | 1 | g |
| MgSO₄•H₂O | 1 | g |
| Sodium citrate | 5 | g |
| Yeast extract | 2 | g |
| CaCO₃ | 40 | g |
| L-Phenylalanine | 0.15 | g |
| L-tyrosine | 0.1 | g |

| | |
|---|---|
| pH | 6.8 |

TABLE 2

| | Production |
|---|---|
| | amount of |
| | L-tryptophan |
| Strain | (mg/L)* |
| W3110 trpΔ2/pCL-Dtrp_att-trpEDCBA | 562 |
| W3110 trpΔ2_ΔfhuC/pCL-Dtrp_att-trpEDCBA | 781 |
| W3110 trpΔ2_ΔfhuD/pCL-Dtrp_att-trpEDCBA | 816 |
| W3110 trpΔ2_ΔfhuB/pCL-Dtrp_att-trpEDCBA | 779 |
| W3110 trpΔ2_ΔhuCDB/pCL-Dtrp_att-trpEDCBA | 796 |

*measured at 48 hours

As shown in Table 2, it was demonstrated that the strains with improved intracellular ATP levels, prepared in Example 3 by deleting any or all of fhuC, fhuD, and fhuB genes in the wild-type L-tryptophan-producing strain W3110 trpΔ2/pCL-Dtrp_att-trpEDCBA, increased L-tryptophan production up to about 63%, compared to the unmodified strain trpΔ2/pCL-Dtrp_att-trpEDCBA. In view of the intracellular ATP levels confirmed in FIG. 2, these results indicate that L-tryptophan producibilities of the strains were increased by the increased intracellular ATP levels.

### Example 5

### Preparation of L-Threonine-Producing Strain and L-Tryptophan Producing Strain Having Inactivation of Proteins Encoded by fhuC, fhuD, and fhuB Genes

In this Example, fhuC, fhuD, and fhuB genes of the L-tryptophan producing strain KCCM10812P (Korean Patent No. 0792095) and the L-threonine producing strain KCCM10541 (Korean Patent No. 0576342) were deleted by homologous recombination, respectively, as in Example 1.

The unmodified strain having L-tryptophan producibility, E. coli KCCM10812P is a strain derived from an E. coli variant (KFCC 10066) having L-phenylalanine producibility, and is a recombinant E. coli strain having L-tryptophan producibility, characterized in that chromosomal tryptophan auxotrophy was desensitized or removed, pheA, trpR, mtr and tnaAB genes were attenuated, and aroG and trpE genes were modified.

Also, the unmodified strain having L-threonine producibility, E. coli KCCM10541P is a strain derived from E. coli KFCC10718 (Korean Patent Publication No. 1992-0008365), and is E. coli having resistance to L-methionine analogue, a methionine auxotroph phenotype, resistance to L-threonine analogue, a leaky isoleucine auxotroph phenotype, resistance to L-lysine analogue, and resistance to α-aminobutyric acid, and L-threonine producibility.

The fhuC, fhuD, and fhuB genes to be deleted were deleted from E. coli KCCM10812P and E. coli KCCM10541P in the same manner as in Example 1, respectively. As a result, an L-threonine producing strain, KCCM10541_ΔfhuCDB and an L-tryptophan producing strain, KCCM10812P_ΔfhuCDB were prepared.

### Example 6

### Measurement of ATP Levels in L-Threonine Producing Strain and L-Tryptophan Producing Strain Having Inactivation of Proteins Encoded by fhuC, fhuD, and fhuB Genes

In this Example, the intracellular ATP levels in the strains prepared in Example 5 were practically measured.

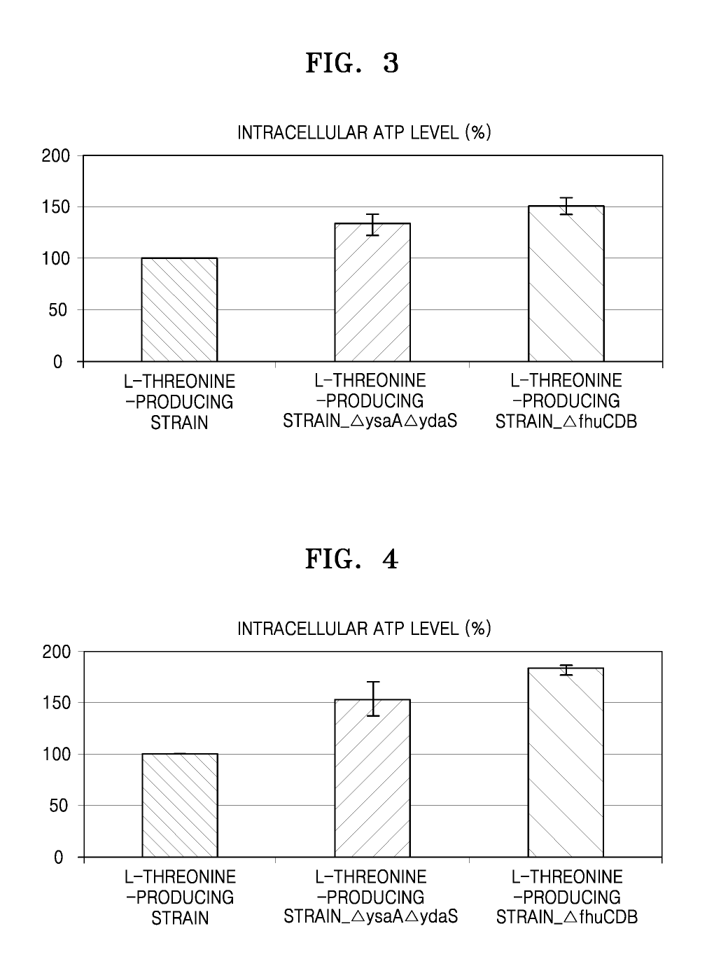

The intracellular ATP levels were measured in the same manner as in Example 2. The results are given in FIGS. 3 and 4. The results of FIGS. 3 and 4 were recorded as the average of three repeated experiments. As control groups, used were ysa and ydaS-deleted, L-threonine producing strain (E. coli KCCM10541P_ΔysaΔydaS) and L-tryptophan producing strain (E. coli KCCM10812P_ΔysaΔydaS) which are known to have higher intracellular ATP levels than the unmodified strains, E. coli KCCM10812P and E. coli KCCM10541P used in Example 3 (Korean Patent No. 1327093).

As shown in FIGS. 3 and 4, fhuC, fhuD, and fhuB-deleted strains prepared from the L-threonine producing strain and the L-tryptophan producing strain in Example 3 showed increased intracellular ATP levels, compared to the unmodified strains and control strains.

### Example 7

### Examination of Titer of L-Threonine-Producing Strain Having the Inactivated Proteins Encoded by fhuC, fhuD, and fhuB Genes

As described in Example 5, the strains with improved intracellular ATP levels, which were prepared by deletion of fhuC, fhuD, and fhuB genes in an L-threonine producing microorganism, E. coli KCCM10541P (Korean Patent No. 0576342), were subjected to titration using glucose as a carbon source. The ysa and ydaS-deleted L-threonine producing strain (E. coli KCCM10541P_ΔysaΔydaS) was used as a control group to compare the titration results.

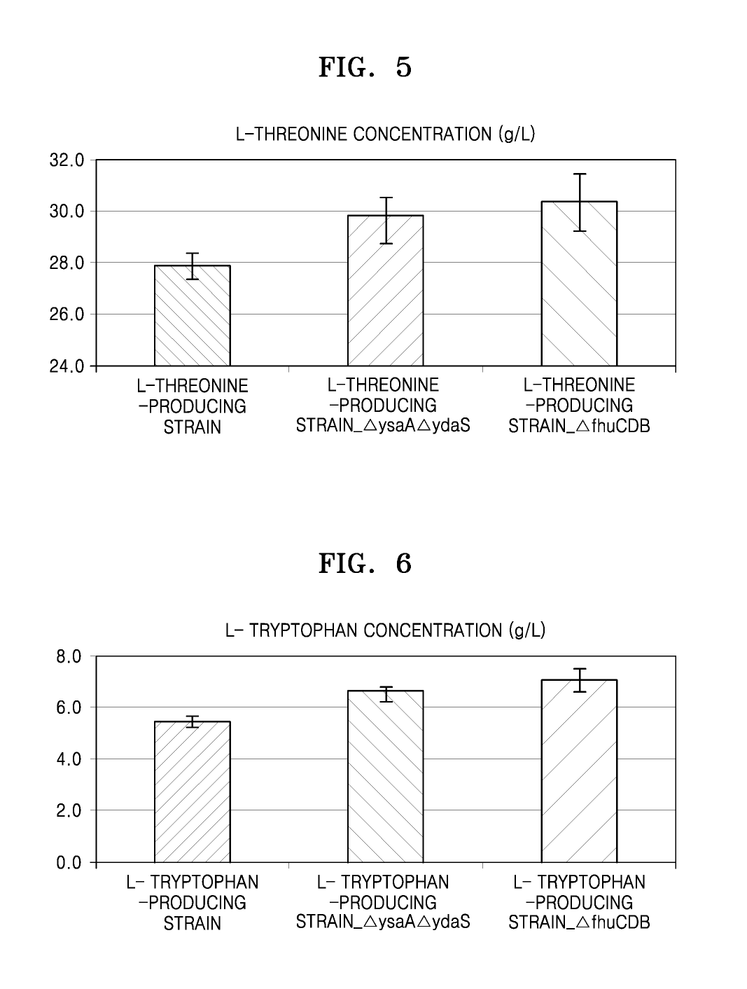

Each of the strains was cultured on an LB solid medium in an incubator at 33° C. overnight, and then inoculated by a platinum loop into 25 mL of a glucose-containing titration medium containing the composition of Table 3. Then, the strains were cultured in an incubator at 33° C. and at 200 rpm for 50 hours. The results are given in Table 4 and FIG. 5. All the results were recorded as the average of three repeated experiments.

TABLE 3

| | Concentration | |
|---|---|---|
| Composition | (per liter) | |
| Glucose | 70 | g |
| KH₂PO₄ | 2 | g |
| (NH₄)₂SO₄ | 25 | g |
| MgSO₄•H₂O | 1 | g |
| FeSO₄•H₂O | 5 | mg |
| MnSO₄•H₂O | 5 | mg |
| Yeast extract | 2 | g |
| CaCO₃ | 30 | g |

TABLE 4

| | | | Production |
|---|---|---|---|
| | | Glucose | amount of |
| | | consumption | L-threonine |
| Strain | OD₅₆₂ | (g/L)* | (g/L)** |
| KCCM10541P | 22.8 | 41.0 | 28.0 ± 0.5 |
| KCCM10541P_ΔysaAΔydaS | 23.9 | 42.1 | 29.8 ± 0.9 |
| KCCM10541P_ΔfhuCDB | 23.1 | 41.8 | 30.5 ± 1 |

*measured at 30 hours
**measured at 50 hours

As shown in Table 4, it was demonstrated that the recombinant L-threonine producing E. coli strain prepared according to the present application showed a physiological activity similar to that of the unmodified strain, and increased L-threonine production up to about 9%, compared to the unmodified strain. In view of the intracellular ATP levels confirmed in FIG. 3, these results indicate that L-threonine producibilities of the strains were increased by the increased intracellular ATP levels.

### Example 8

### Examination of Titer of L-Tryptophan-Producing Strain Having Inactivation of Proteins Encoded by fhuC, fhuD, and fhuB Genes

As described in Example 5, the strains with improved intracellular ATP levels, which were prepared by deletion of fhuC, fhuD, and fhuB genes in an L-tryptophan producing microorganism, KCCM10812P (Korean Patent No. 0792095), were subjected to titration using glucose as a carbon source. The ysa and ydaS-deleted L-tryptophan producing strain (E. coli KCCM10812P_ΔysaΔydaS) was used as a control group to evaluate the titer in the same manner as in Example 4.

The titration results are given in Table 5 and FIG. 6. All the results were recorded as the average of three repeated experiments.

TABLE 5

| | | | Production |
|---|---|---|---|
| | | Glucose | amount of |
| | | consumption | L-tryptophan |
| Strain | OD₆₀₀ | (g/L)* | (g/L)** |
| KCCM10812P | 18.2 | 45.7 | 5.5 ± 0.2 |
| KCCM10812P_ΔysaAΔydaS | 18.3 | 46.3 | 6.7 ± 0.1 |
| KCCM10812P_ΔfhuCDB | 17.9 | 47.4 | 7.1 ± 0.5 |

*measured at 33 hours
**measured at 48 hours

As shown in Table 5, it was demonstrated that the recombinant L-tryptophan producing E. coli strain prepared according to the present application showed a physiological activity similar to that of the unmodified strain, and increased L-tryptophan production up to about 30%, compared to the unmodified strain. In view of the intracellular ATP levels confirmed in FIG. 4, these results indicate that L-tryptophan producibilities of the strains were increased by the increased intracellular ATP levels.

The recombinant strain of the present application, CA04-2801 (KCCM10812P_ΔfhuCDB) was deposited at the Korean Culture Center of Microorganisms, an international depository authority, on Nov. 15, 2013 under Accession NO. KCCM11474P.

Based on the above description, it will be understood by those skilled in the art that the present application may be implemented in a different specific form without changing the technical spirit or essential characteristics thereof. Therefore, it should be understood that the above embodiment is not limitative, but illustrative in all aspects. The scope of the application is defined by the appended claims rather than by the description preceding them, and therefore all changes and modifications that fall within metes and bounds of the claims, or equivalents of such metes and bounds are therefore intended to be embraced by the claims.

1. A microorganism of the genus Escherichia having an increased intracellular ATP level, compared to an unmodified strain, wherein activities of one or more proteins selected from an amino acid sequence of SEQ ID NO: 5, an amino acid sequence of SEQ ID NO: 6, and an amino acid sequence of SEQ ID NO: 7, which constitute an iron uptake system, were inactivated.

2. The microorganism of the genus Escherichia according to claim 1, wherein the activities of all of the proteins having amino acid sequences of SEQ ID NOS: 5, 6, and 7 were inactivated.

3. The microorganism of the genus Escherichia according to claim 1, wherein the microorganism is E. coli.

4. The microorganism of the genus Escherichia according to claim 1, wherein the microorganism of the genus Escherichia has an improved ability to produce L-amino acid, compared to an unmodified strain.

5. The microorganism of the genus Escherichia according to claim 4, wherein the L-amino acid is L-threonine or L-tryptophan.

6. A method for producing L-amino acids, the method comprising:
culturing the microorganism of the genus Escherichia of claim 1 in a media, and
recovering L-amino acids from the culture media or the microorganism.

7. The method of claim 6, wherein the L-amino acid is L-threonine or L-tryptophan.

8. A method for producing L-amino acids, the method comprising:
culturing the microorganism of the genus Escherichia of claim 2 in a media, and
recovering L-amino acids from the culture media or the microorganism.

9. A method for producing L-amino acids, the method comprising:
culturing the microorganism of the genus Escherichia of claim 3 in a media, and
recovering L-amino acids from the culture media or the microorganism.

10. A method for producing L-amino acids, the method comprising:
culturing the microorganism of the genus Escherichia of claim 4 in a media, and
recovering L-amino acids from the culture media or the microorganism.

11. A method for producing L-amino acids, the method comprising:
culturing the microorganism of the genus Escherichia of claim 5 in a media, and
recovering L-amino acids from the culture media or the microorganism.

12. The method of claim 8, wherein the L-amino acid is L-threonine or L-tryptophan.

13. The method of claim 9, wherein the L-amino acid is L-threonine or L-tryptophan.

14. The method of claim 10, wherein the L-amino acid is L-threonine or L-tryptophan.

15. The method of claim 11, wherein the L-amino acid is L-threonine or L-tryptophan.
